(19) United States
(12) Patent Application Publication
(10) Pub. No.: US 2008/0058398 A1
(43) Pub. Date: Mar. 6, 2008

(54) Compounds

(76) Inventors: Clare Louise Anderton, Stevenage (GB); Sergio Bacchi, Verona (IT); Stefania Beato, Verona (IT); Franco Sartor, Verona (IT)

Correspondence Address: GLAXOSMITHKLINE;Corporate Intellectual Property - UW2220, P.O. Box 1539, King of Prussia, PA, 19406-0939, US

(21) Appl. No.: 11/841,015

(22) Filed: Aug. 20, 2007

(30) Foreign Application Priority Data
Aug. 21, 2006 (GB) 0616574.0
Aug. 21, 2006 (EP) PCT/EP2006/008314

(51) Int. Cl. A61K 31/422; A61P 25/18; A61P 25/22; A61P 25/24; A61P 25/30; A61P 25/32; A61P 25/34; A61P 25/36; C07D 413/14

(52) U.S. Cl. 514/374; 548/235

(57) ABSTRACT

The present invention relates to the tartrate salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane and solvates thereof, pharmaceutical formulations, processes for their preparation, and their use in medicine.

### FIELD OF THE INVENTION

The present invention relates to a novel salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane and solvates thereof, pharmaceutical formulations, processes for their preparation, and their use in medicine.

### BACKGROUND OF THE INVENTION

The structure of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane is indicated below as the compound of formula (I): 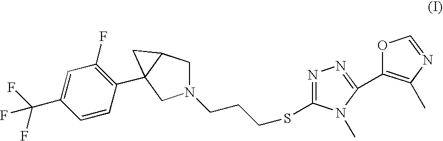
The compound of formula (I) can be prepared by the reaction of 1-[2-fluoro-4-(trifluoromethyl)phenyl]-3-azabicyclo[3.1.0]hexane and 3-[(3-chloropropyl)thio]-4-methyl-5-methyl-1,3-oxazol-5-yl)-4H-1,2,4-triazole, in a suitable solvent, for example DMF or NMP, in the presence of a base, for example Na₂CO₃ or K₂CO₃ in combination with NaI or KI.

The hydrochloride salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane can be prepared by adding hydrochloric acid or hydrogen chloride under N₂ to a solution of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane in an etheral solvent (such as Et₂O) or an alcoholic solvent (such as isopropanol).

The compound of formula (I) and its hydrochloride salt, described in the International Patent Application WO2005/080382, have been found to be useful in the treatment of all aspects of drug dependency, including drug intake, relapse to drug-seeking behaviour following abstinence and withdrawal symptoms from drugs of abuse such as alcohol, cocaine, opiates, nicotine, benzodiazepines and inhibition of tolerance induced by opioids, as well as for the treatment of drug craving. It is also useful as an antipsychotic agent for example in the treatment of schizophrenia, schizo-affective disorders, schizophreniform diseases, psychotic depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion), anxiety disorders (which includes generalised anxiety and social anxiety disorder), mania, acute mania, paranoid and delusional disorders. The compounds are also useful for the treatment of a family of related disorders referred to as somatoform disorders, as well as for the treatment of premature ejaculation.

For use in medicine there exists a need for the compound to be prepared in a form suitable for ease of isolation in large scale manufacture and ease of formulating into an acceptable product for administration to patients. It is difficult to predict the physical characteristics of any particular salt of a compound and small, but significant, differences in physical properties may equate to large savings in the manufacture and formulation of a pharmaceutical product containing the compound.

### SUMMARY OF THE INVENTION

The present invention provides 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, which may be used as an alternative to the free base and the hydrochloride salt of the compound of formula (I) for therapeutic administration or as an intermediate in the preparation of other salts.

### DESCRIPTION OF THE FIGURES

FIG. 1 shows X-Ray powder diffraction data obtained for (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate.

FIG. 2 shows the Raman spectrum of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate prepared by a first process.

FIG. 3 shows a Differential Scanning Calorimetry (DSC) thermogram of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate.

FIG. 4 shows X-Ray powder diffraction data obtained for (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate prepared by an alternative process to that of the material in FIG. 1.

FIG. 5 shows a Raman spectrum of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate prepared by a second process.

FIG. 6 shows a second Differential Scanning Calorimetry (DSC) thermogram of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate.

FIG. 7 shows a Carbon-13 solid State NMR spectrum performed on a different sample of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate.

The tartrate salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane may be prepared by an efficient, economic and reproducible process particularly suited to large scale preparation.

The tartrate salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (hereinafter also referred to as “the tartrate”) has improved stability over the hydrochloride salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane.

Therefore, as a first aspect of the present invention there is provided 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof.

The present invention includes within its scope all isomers, including racemates, enantiomers, tautomers and mixtures thereof. For example, it will be appreciated that tartaric acid (HO₂C—CH(OH)—CH(OH)—CO₂H; 2,3-dihydroxybutanedioic acid) exists in three stereoisomeric configurations: (2R,3R), which is naturally occurring and is also known as L-(+)-tartaric acid or dextrotartaric acid; (2S,3S) which is known as levotartaric acid or D-(−)-tartaric acid; and the achiral form, mesotartaric acid. The present invention encompasses the tartrate salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane derived from all three stereoisomeric configurations of tartaric acid. As used herein, the terms “tartrate” and “tartaric acid” are intended to include all stereoisomeric configurations unless otherwise indicated.

In one embodiment, the present invention provides 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane-(2R,3R) tartrate or a pharmaceutically acceptable solvate thereof.

It will also be appreciated that 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane possesses chiral centres at positions 1 and 5 in the 3-azabicyclo[3.1.0]hexane portion of the molecule. Because of the fixed cis disposition, the compound exists in two stereoisomers which are enantiomers with respect to the chiral centres in the cyclopropane:

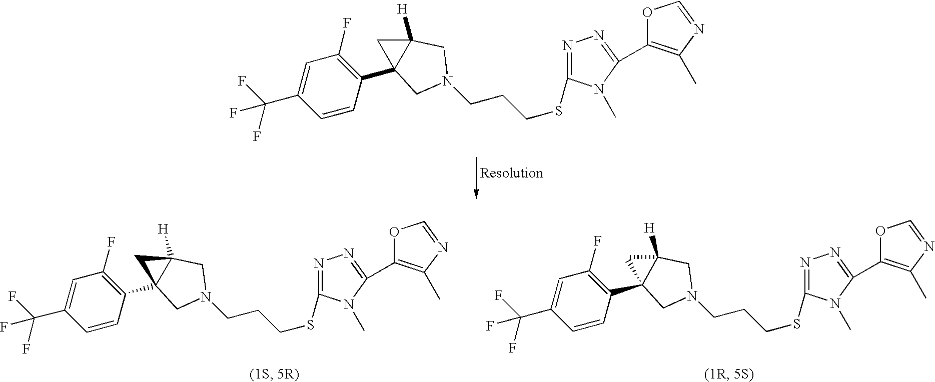

In one embodiment of the present invention, there is provided (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof.

As used herein, the term “1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate” encompasses:
(i) (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate;
(ii) (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2S,3S) tartrate;
(iii) (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (meso) tartrate;
(iv) (1R,5S)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate;
(v) (1R,5S)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2S,3S) tartrate;
(vi) (1R,5S)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (meso) tartrate;
(vii) a mixture of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate and (1R,5S)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate;
(viii) a mixture comprising any combination of (i), (ii), (iii), (iv), (v) and/or (vi) as defined above.

As used herein, the term “(1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate” encompasses:
(ix) (1S,5R)-1-[2-fluoro-4-(trifluoromethyl)phenyl]-3-(3-{[4-methyl-5-(4-methyl-5-(4-yl)-4H-1,2,4-triazol-3-yl]thio}propyl)-3-azabicyclo[3.1.0]hexane (2R,3R) tartrate;
(x) (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2S,3S) tartrate;
(xi) (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (meso) tartrate;
(xii) a mixture comprising any combination of (ix), (x) and/or (xi) as defined above.

In the context of the present invention, stereochemical isomers enriched in configuration (1S,5R) of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate correspond in one embodiment to at least 90% enantiomeric excess. In another embodiment, the isomers correspond to at least 95% enantiomeric excess. In another embodiment, the isomers correspond to at least 99% enantiomeric excess.

In another aspect of the invention, there is provided 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate in which the ratio of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane to tartaric acid (by mole) is 1:1.

In one embodiment of the present invention, the tartrate is substantially free of alternative salt, free base or impurity. By “substantially free” is meant containing less than 10%, preferably less than 5%, more preferably less than 2%, of impurity. The impurity may be other compounds or other salts or solvates of the compound of formula (I).

Depending on the solvent from which the tartrate is recovered, the tartrate may be obtained as a solvate, such a solvate also forms one aspect of the present invention. In one embodiment the solvate is a pharmaceutically acceptable solvate. A suitable solvate is a hydrate. In a further embodiment the hydrate may have a variable water content between 2-5% wt/wt. In one embodiment, 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate which is a sesquihydrate (1:1.5 molecules of water) is provided.

The present invention encompasses 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof isolated in pure form or when admixed with other materials.

Therefore, in one aspect there is provided 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof in isolated form.

In another aspect there is provided 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof in pure form. In one embodiment, 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate is greater than 90% pure, such as greater than 95% pure, or greater than 98% pure.

In a further aspect there is provided (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof in crystalline form.

In a still further aspect there is provided 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof in polymorphic form(s).

A further aspect of the invention provides (1S,5R)-1-[2-fluoro-4-(trifluoromethyl)phenyl]-3-(3-{[4-methyl-{[4-methyl-5-(4-methyl-1,3-oxazol-5-yl)-4H-1,2,4-triazol-3-yl]thio}propyl)-3-azabicyclo[3.1.0]hexane (2R,3R) tartrate having a melting point of 122° C. and having a Raman or XRPD spectrum or C13 solid state NMR spectrum substantially as disclosed below.

The present invention also provides for 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a solvate thereof when admixed with other material, for example another salt of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane.

The 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may be prepared by contacting appropriate stoichiometric amounts of free base with tartaric acid. In one embodiment, the base is in solution. In another embodiment, both are in solution.

Most commonly used solvents are suitable for mobilising the free base, for example alcohols such as ethanol or methanol, ketones such as acetone, esters such as ethyl acetate, halogenated hydrocarbons such as dichloromethane, and ethers such as tetrahydrofuran. The tartaric acid may be added as a solid, as an aqueous solution, or as a solution in an organic solvent such as ethanol, methanol, propan-2-ol, or acetone.

For the preparation of the tartrate, the concentration of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo-[3.1.0]hexane base is preferably in the range 3 to 25% weight/volume, more preferably in the range 5 to 15%. The concentration of tartaric acid when used in solution is preferably in the range 0.5 to 10 molar, such as for example between 5 to 10 molar.

The salt may be isolated in solid form by conventional means from a solution thereof obtained as above. For example, a non-crystalline salt may be prepared by precipitation from solution, spray drying and freeze drying of solutions, evaporating a solution to a glass, or vacuum drying of oils, or solidification of melts obtained from reaction of the free base and the acid.

Crystalline salts may be prepared by directly crystallising from a solvent in which the product has limited solubility, or by triturating or otherwise crystallising a non-crystalline salt. For example, the tartrate may be recrystallised from a variety of organic solvents, such as acetonitrile, butanone, acetone, sec-butanol, dichloromethane, ethanol, 3-pentanone, propan-2-ol, methanol, ethyl acetate and toluene. An improved yield of the salt is obtained by evaporation of some or all of the solvent or by crystallisation at elevated temperature followed by controlled cooling, preferably in stages. Careful control of precipitation temperature and seeding may be used to improve the reproducibility of the production process and the particle size distribution and form of the product. Individual polymorphs are preferably crystallized directly from a solution of the salt, although recrystallizing a solution of one polymorph using seeds of another polymorph may also be carried out.

1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane may be prepared by the process set forth in the Examples.

1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may be obtained as a solvate, when during isolation from solution it becomes associated with the solvent in which it is dissolved.

Tartaric acid is commercially available.

The present invention further provides a process for preparing (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate, according to the following Scheme 1, which will be illustrated in the Experimental Section.

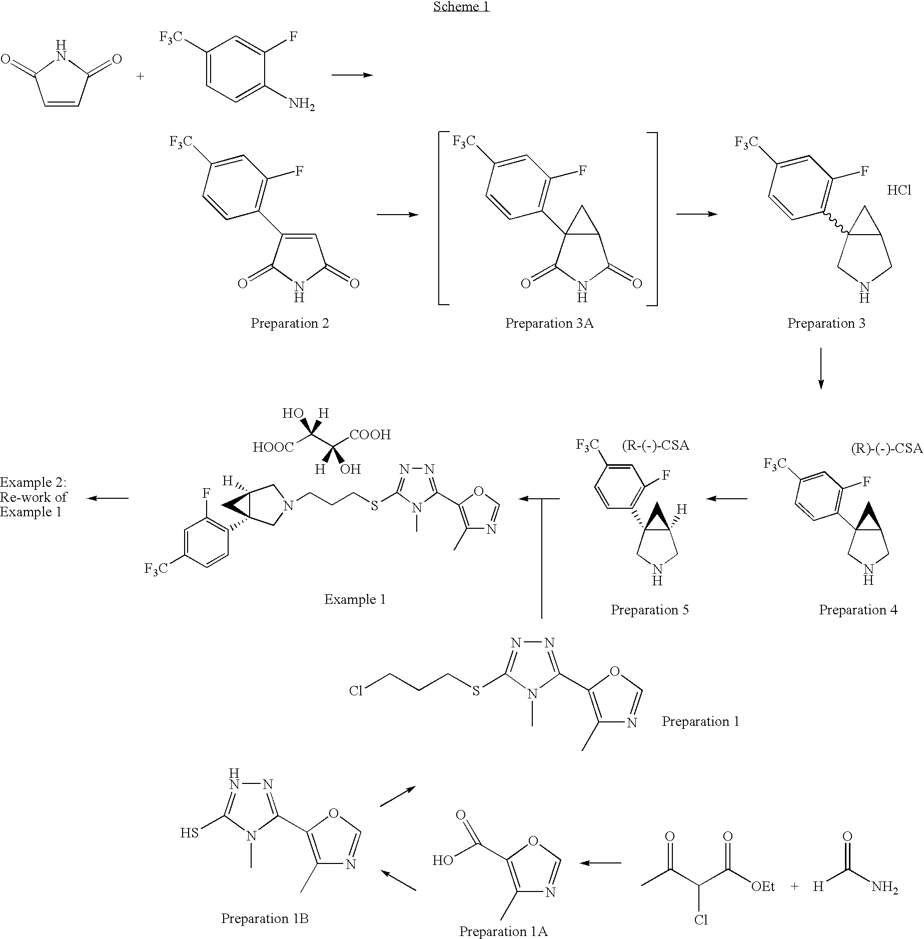

(1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane has been found to exhibit affinity for dopamine receptors, in particular the D₃ and D₂ receptors, and is useful in the treatment of disease states which require modulation of such receptors. (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane has also been found to have greater affinity for dopamine D₃ than for D₂ receptors.

From the localisation of D₃ receptors, it could be envisaged that the tartrate would have utility for the treatment of a substance-related disorder where it has been suggested that D₃ receptors are involved (e.g. see Levant, 1997, Pharmacol. Rev., 49, 231-252; and Heidbreder C A, Gardner E L, Xi Z X, Thanos P K, Mugnaini M, Hagan J J, Ashby C R Jr. (2005) Brain Res. Brain Res. Rev., 49(1): 77-105). Examples of such substance abuse are cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine or methylamphetamine abuse or a combination thereof. 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may be used for treatment of all aspects of drug dependency including drug intake, relapse to drug-seeking behaviour following abstinence and withdrawal symptoms from drugs of abuse such as alcohol, cocaine, opiates, nicotine, benzodiazepines and inhibition of tolerance induced by opioids. In addition, 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may be used to reduce craving and therefore will be useful in the treatment of drug craving. Drug craving can be defined as the incentive motivation to self-administer a psychoactive substance that was previously consumed. Three main factors are involved in the development and maintenance of drug craving: (1) Dysphoric states during drug withdrawal can function as a negative reinforcer leading to craving; (2) Environmental stimuli associated with drug effects can become progressively more powerful (sensitization) in controlling drug seeking or craving, and (3) A cognition (memory) of the ability of drugs to promote pleasurable effects and to alleviate a dysphoric state during withdrawal. Craving may account for the difficulty that individuals have in giving up drugs of abuse and therefore contributes significantly to the development and maintenance of drug dependence.

The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. It has been suggested that blockade of the recently characterised dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314,1993).

Thus 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate is of potential use as antipsychotic agents for example in the treatment of schizophrenia, schizo-affective disorders, psychotic depression, mania, paranoid and delusional disorders. Furthermore, they could have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). Other conditions which may be treated by the tartrate include dyskinetic disorders such as Parkinson's disease, neuroleptic-induced parkinsonism and tardive dyskinesias; depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion); anxiety disorders (which includes generalised anxiety and social anxiety disorder); agitation; tension; social or emotional withdrawal in psychotic patients; cognitive impairment including memory disorders (including Alzheimer's disease, dementia, amnesic disorders and age-associated memory impairment); psychotic states associated with neurodegenerative disorders, e.g. Alzheimer's disease; eating disorders (including anorexia nervosa and bulimia nervosa); obesity; sexual dysfunction; sleep disorders (including disturbances of circadian rhythm, dyssomnia, insomnia, sleep apnea and narcolepsy); emesis; movement disorders; obsessive-compulsive disorders; amnesia; aggression; autism; vertigo; dementia; circadian rhythm disorders; convulsions; epilepsy; and gastric motility disorders e.g. IBS.

A wide range of psychiatric and neuropsychiatric disorders appear to be related to Obsessive-Compulsive Disorder, and in particular, somatoform disorder 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may also be used for the treatment of a somatoform disorder such as body dysmorphic disorder and hyperchondriasis, bulimia nervosa, anorexia nervosa, binge eating, paraphilia and nonparaphilic sexual addictions, Sydeham's chorea, torticollis, autism, and movement disorders, including Tourette's syndrome.

1-[2-fluoro-4-(trifluoromethyl)phenyl]-3-(3-{[4-methyl-5-(4-methyl-1,3-oxazol-5-yl)-4-1,24-triazol-3-yl]thio}propyl)-3-azabicyclo[3.1.0]hexane tartrate is also useful for the treatment of premature ejaculation.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “substance-related disorder” includes:-

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

Within the context of the present invention, the term “psychotic disorder” includes:-

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Therefore, the invention provides 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof for use in therapy. In particular the invention provides 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof for use in the treatment of a condition which requires modulation of a dopamine receptor, such as treatment of a substance-related disorder.

The invention also provides 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof for use in the treatment of a somatoform disorder.

The invention also provides the use of 1-[2-fluoro-4-(trifluoromethyl)phenyl]-3-(3-{[4-methyl-5-4-methyl-1,3-oxazol-5-yl)-4H-1,2,4-triazol-3-yl]thio}propyl)-3-azabicyclo[3.1.0]hexane tartrate or a pharmaceutically acceptable solvate thereof in the manufacture of a medicament for the treatment of a condition which requires modulation of a dopamine receptor. In particular, the invention provides the use of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof in the manufacture of a medicament for the treatment of a substance-related disorder.

The invention also provides the use of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof in the manufacture of a medicament for the treatment of a psychotic disorder or a somatoform disorder.

The invention also provides a method of treating a condition which requires modulation of a dopamine receptor, which comprises administering to a mammal in need thereof an effective amount of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof. In particular, the invention provides a method of treating a substance-related disorder, which comprises administering to a mammal in need thereof an effective amount of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof.

The invention also provides a method of treating a psychotic disorder or a somatoform disorder, which comprises administering to a mammal in need thereof an effective amount of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof.

“Treatment” includes prophylaxis, where this is appropriate for the relevant condition(s).

For use in medicine, 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate is usually administered as a standard pharmaceutical composition. The present invention therefore provides in a further aspect a pharmaceutical composition comprising 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate and a pharmaceutically acceptable carrier. The pharmaceutical composition can be for use in the treatment of any of the conditions described herein.

1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate may be administered by any convenient method, for example by oral, parenteral (e.g. intravenous), buccal, sublingual, nasal, rectal or transdermal administration and the pharmaceutical compositions adapted accordingly.

1-[2-fluoro-4-(trifluoromethyl)phenyl]-3-(3-{[4-methyl-5-(4-methyl-1,3-oxazol-4-yl)-4H-1,2,4-triazol-3-yl]thio}propyl)-3-azabicyclo[3.1.0]hexane tartrate can be formulated as liquids or solids, for example syrups, suspensions or emulsions, tablets, capsules and lozenges.

A liquid formulation will generally consist of a suspension or solution of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastlles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. Preferably the composition is in unit dose form such as a tablet, capsule or ampoule.

Each dosage unit for oral administration contains preferably from 1 to 250 mg (and for parenteral administration contains preferably from 0.1 to 25 mg) of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate calculated as the free base. 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate will normally be administered in a daily dosage regimen (for an adult patient) of, for example, an oral dose of between 1 mg and 500 mg, such as between 25 mg and 500 mg, e.g. between 55 and 280 mg or an intravenous, subcutaneous, or intramuscular dose of between 0.1 mg and 100 mg, such as between 0.1 mg and 50 mg, e.g. between 1 and 25 mg of the compound of the formula (I) calculated as the free base, the compound being administered 1 to 4 times per day. Suitably the compounds will be administered for a period of continuous therapy, for example for a week or more.

No toxicological effects are expected when a compound of the invention is administered in the above mentioned dosage range.

The invention is further illustrated by the following non-limiting examples.

Biological Test Methods

Functional potency and intrinsic activity of compounds of this invention can be measured by the following GTPγS scintillation proximity assay (GTPγS-SPA). Cells used in the study are Chinese Hamster Ovary (CHO) Cells.

Cell Line

CHO_D2

CHO_D3

Cell membranes are prepared as follows. Cell pellets are resuspended in 10 volumes of 50 mM HEPES, 1 mM EDTA pH 7.4, using KOH. On the day the following proteases are added to the buffer just prior to giving the homogenisation buffer.

10⁻⁶M Leupeptin (Sigma L2884)−5000×stock=5 mg/mL in buffer

25 ug/mL Bacitracin (Sigma B0125)−1000×stock=25 mg/mL in buffer

1 mM PMSF−1000×stock=17 mg/mL in 100% ethanol

2×10⁻⁶M Pepstain A−1000×stock=2 mM in 100% DMSO

The cells are homogenised by 2×15 second bursts in a 1 litre Glass Waring blender in a class two biohazard cabinet. The resulting suspension is spun at 500 g for 20 mins (Beckman T21 centrifuge: 1550 rpm). The supernatant is withdrawn with a 25 mL pipette, aliquotted into pre-chilled centrifuge tubes and spun at 48,000 g to pellet membrane fragments (Beckman T1270: 23,000 rpm for 30 mins). The final 48,000 g pellet is resuspended in Homogenisation Buffer, (4× the volume of the original cell pellet). The 48,000 g pellet is resuspended by vortexing for 5 seconds and homogenized in a dounce homogenizer 10-15 stokes. The prep is distributed into appropriate sized aliquots, (200-1000 ul), in polypropylene tubes and store at −80° C. Protein content in the membrane preparations is evaluated with the Bradford protein assay.

The final top concentration of test drug is 3 uM in the assay and 11 points serial dilution curves 1:4 in 100% DMSO are carried out using a Biomek FX. The test drug at 1% total assay volume (TAV) is added to a solid, white, 384 well assay plate. 50% TAV of precoupled (for 90 mins at 4° C.) membranes, 5 μg/well, and Wheatgerm Agglutinin Polystyrene Scintillation Proximity Assay beads (RPNQO260, Amersham), 0.25 mg/well, in 20 mM HEPES pH 7.4, 100 mM NaCl, 10 mM MgCl₂, 60 μg/mL saponin and 30 μM GDP is added. The third addition was a 20% TAV addition of either buffer, (agonist format) or EC₈₀ final assay concentration of agonist, Quinelorane, prepared in assay buffer (antagonist format). The assay was started by the addition of 29% TAV of GTPγ[³⁵S] 0.38 nM final (37 MBq/mL, 1160 Ci/mmol, Amersham). After all additions assay plates are spun down for 1 min at 1,000 rpm. Assay plates are counted on a Viewlux, 613/55 filter, for 5 min., between 2-6 hours after the final addition.

The effect of the test drug over the basal generates EC₅₀ value by an iterative least squares curve fitting programme, expressed in the table as pEC₅₀ (i.e. −logEC₅₀). The ratio between the maximal effect of the test drug and the maximal effect of full agonist, Quinelorane, generates the Intrinsic Activity (IA) value (i.e. IA=1 full agonist, IA<1 partial agonist). fpKi values of test drug are calculated from the IC₅₀ generated by “antagonist format” experiment, using Cheng & Prusoff equation: fKi=IC₅₀/1+([A]/EC₅₀) where: [A] is the concentration of the agonist 5-HT in the assay and EC₅₀ is the 5-HT EC₅₀ value obtained in the same experiment. fpKi is defined as −logfKi.

### EXAMPLES

In the Examples unless otherwise stated:

All temperatures refers to ° C.

Infrared spectra were measured on a FT-IR instrument.

Compounds were analysed by direct infusion of the sample dissolved in acetonitrile into a mass spectra operated in positive electro spray (ES⁺) ionisation mode.

Proton Magnetic Resonance (¹H-NMR) spectra were recorded at 400 MHz, chemical shifts are reported in ppm downfield (d) from Me₄Si, used as internal standard, and are assigned as singlets (s), broad singlets (bs), doublets (d), doublets of doublets (dd), triplets (t), quartets (q) or multiplets (m).

Column chromathography was carried out over silica gel (Merck AG Darmstaadt, Germany). The following abbreviations are used in the text: T3P=N-propane Phosphonic Cyclic Anhydride; DMSO=dimethylsulfoxide.

HPLC Methods

HPLC Assay (Short Run):

| Column type | Phenomenex LUNA | | |
|---|---|---|---|
| Column length [cm] | 5 | | |
| Internal diameter [cm] | 0.2 | | |
| Particle size [um] | 3.0 | | |
| Mobile phase | A: 0.05% v/v TFA in | | |
| | water/B: 0.05% v/v TFA in | | |
| | acetonitrile | | |
| Step 1: Time-Reserv.A-Reserv.B | Time 0 min | 100% | A |
| Step 2: Time-Reserv.A-Reserv.B | Time 8 min | 5% | A |
| Step 3: Time-Reserv.A-Reserv.B | Time 8.01 min | 100% | A |

| | |
|---|---|
| Flow rate [mL/min] | 1 |
| Column temperature [° C.] | 40 |
| Autosampler temperature [\hat{ }C.] | AMB |
| Detector type | UV |
| Wavelength [nm] | 220 |
| Injection volume [uL] | 1 |
| Run Time | 8 min. |

HPLC Chiral 1

| Column type | Chiracel OD-H |
|---|---|
| Column length [cm] | 25 |
| Internal diameter [cm] | 4.6 |
| Particle size [um] | 5 |
| Mobile phase | Heptane/IPA 85/15% v/v |
| Flow rate [mL/min] | 1 |
| Column temperature [\hat{ }C.] | 30 |
| Autosampler temperature [\hat{ }C.] | AMB |
| Detector type | UV |
| Wavelength [nm] | 220 |
| Injection volume [uL] | 10 |
| Dilution Factor | 5 |

HPLC Assay (Long Run):

| Column type | LUNA 3u phenyl - hexyl | |
|---|---|---|
| Column length [cm] | 15 | |
| Internal diameter [cm] | 0.46 | |
| Particle size [um] | 3.0 | |
| Mobile phase | A: 0.05% v/v TFA in water/ | |
| | B: 0.05% v/v TFA in | |
| | acetonitrile | |
| Step 1: Time-Reserv.A-Reserv.B | Time 0 min | 95% A-5% B |
| Step 2: Time-Reserv.A-Reserv.B | Time 30 min | 5% A-95% B |
| Step 3: Time-Reserv.A-Reserv.B | Time 30.01 min | 95% A-5% B |

| | |
|---|---|
| Flow rate [mL/min] | 1 |
| Column temperature [\hat{ }C.] | 40 |
| Autosampler temperature [\hat{ }C.] | AMB |
| Detector type | UV |
| Wavelength [nm] | 220 |
| Injection volume [uL] | 10 |
| Run Time | 30 min. |

HPLC Chiral 2

| Column type | CHIRALPAK AD |
|---|---|
| Column length [cm] | 25 |
| Internal diameter [cm] | 4.6 |
| Particle size [um] | 10 |
| Mobile phase | Heptane/IPA 85/15% v/v |
| Flow rate [mL/min] | 0.8 |
| Column temperature [\hat{ }C.] | 25 |
| Autosampler temperature [\hat{ }C.] | AMB |
| Detector type | UV |
| Wavelength [nm] | 270 |
| Injection volume [uL] | 10 |
| Dilution Factor | 10 |

### Preparation 1: 3-[(3-Chloropropyl)thio]-4-methyl-5-(4-methyl-1,3-oxazol-5-yl)-4H-1,2,4-triazole

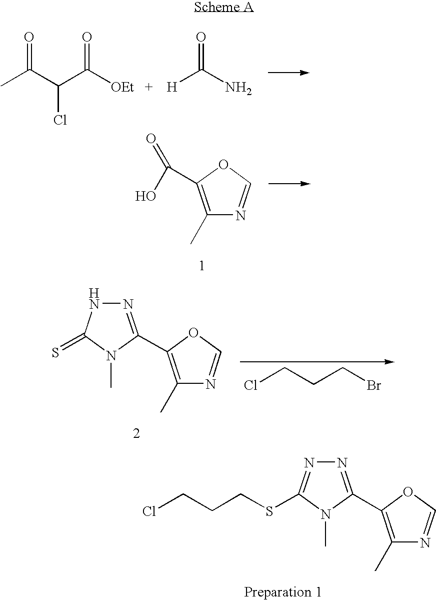

### Preparation 1A: 4-methyl-1,3-oxazole-5-carboxylic acid

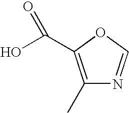

Ethyl-2-chloroacetoacetate (28.6 g, 24.0 mL) was dissolved in DMF (28.6 mL) and formamide (19.5 mL) was added. The resulting solution was heated up to 120° C. (internal temperature) under nitrogen for 21 h. The mixture was allowed to cool down to 20° C., diluted with ter-butyl methyl ether (172 mL) and washed with water (115 mL). The aqueous phase was extracted again with 115 mL of tert-butyl methyl ether and the combined organic layers were washed twice with water (86 mL) and treated with NaOH 3 N (86 mL). The resulting mixture was stirred at 20° C. for 3 hours. The organic layer was discarded while the aqueous was acidified with 20 mL of concentrated HCl (37% sol.) till pH 2 over 10 minutes. A precipitate started to crush out of solution. The suspension was stirred at 20° C. for 2 h, filtered and the cake washed with 14.3 mL of cold water (10° C. ca.). The collected solid was dried under high vacuum at 40° C. for 16 hours. The title compound was obtained in a theoretical yield of 35.5% (7.8 g).

NMR (1H, DMSO-d6, δ ppm): 13.5 (bs, 1H), 8.47 (s, 1H), 2.38 (s, 3H)

MS (m/z): 128[MH]⁺

### Preparation 1B: 4-methyl-5-(4-methyl-1,3-oxazol-5-yl)-2,4-dihydro-3H-1,2,4-triazole-3-thione

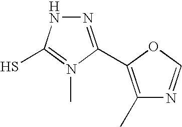

4-Methyl-1,3-oxazole-5-carboxylic acid (prepared according to the method of Preparation 1A, 12.9 g) was dissolved in DMF (60 mL) and treated with 4-methyl-3-thiosemicarbazide (11.61 g). Then disopropylethylamine (DIPEA) (31 mL) was added at 20° C. Under ice bath cooling, T3P 50% w/w in ethyl acetate (90 mL) was added drop wise, maintaining the temperature below 15° C. over 20 minutes. The resulting mixture was then stirred at 20° C. for 6 hours. The mixture was diluted with NaOH 4 M (120.0 mL). The resulting bi-phasic mixture was allowed separating and the upper organic layer discarded. The aqueous layer (pH=8) was adjusted to pH=11 with additional NaOH 4 M (60 mL) and then heated to 70° C. (internal temperature) for 30 minutes. After cooling down over night, HCl 37% was slowly added until pH=5 was reached.

The suspension was stirred for 8 hours, then the solid was filtered and washed with water (60 mL), and it was dried in a vacuum oven at 40° C. overnight. The title compound was obtained in a 53% theoretical yield (10.48 g).

NMR (1H, DMSO-d6, δ ppm): 14.11 (bs, 1H), 8.60 (s, 1H), 3.61 (s, 3H), 2.33 (s, 3H)

MS (m/z): 197[MH]⁺

### 3-[(3-chloropropyl)thio]-4-methyl-5-(4-methyl-1,3-oxazol-5-yl)-4H-1,2,4-triazole

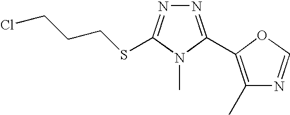

4-methyl-5-(4-methyl-1,3-oxazol-5-yl)-2,4-dihydro-3H-1,2,4-triazole-3-thione (prepared according to the method of Preparation 2A, 380 g) was added to a mixture of methanol (1140 mL) and acetone (2660 mL), followed by K₂CO₃ (380 g) and 1-bromo-3-chloropropane (251 mL). The suspension was stirred at 20° C. for 4 h. The volume of solvent was reduced then ethyl acetate (3800 mL) was added and the organic layer was washed with water twice (2400 mL each). The organic layer was distilled to about 3300 mL, diluted with ethyl acetate (4800 mL) and distilled again to the same level as before. Some precipitate was already observed when cooling the mixture that was stirred for 30 minutes. Heptane (4800 mL) was added slowly over a period of 30 minutes upon which more product crashed out as a fine, heavy solid. The suspension was stirred for four additional hours at 20±2° C. The solid was collected by filtration and washed with 1140 mL of a ethyl acetate/ heptane (1:2) mixture. The solid was dried in the oven at 40° C. under reduced pressure overnight to give the title compound in a 59.3 theoretical yield (314 g).

NMR (1H, DMSO-d6, δ ppm): 8.55 (s, 1H), 3.76 (t, 2H), 3.68 (s, 3H), 3.26 (t, 2H), 2.37 (s, 3H), 2.14 (m, 2H)

MS (m/z): 273[MH]⁺

### Preparation 2: 3-[2-fluoro-4-(trifluoromethyl)phenyl]-1H-pyrrole-2,5-dione

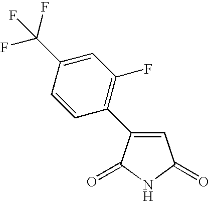

Maleimide (48.6 g) was suspended in acetonitrile (300 mL,) under N₂ and tert-butyl nitrite (38 mL) followed by copper (II) chloride (45 g) were added. The resulting suspension was cooled down to 0° C. and neat 4-amino-2-fluorotrifluorobenzene (50 g, 35.2 mL) was added drop wise in 45 min ca. The internal temperature was kept below 10° C. during the aniline addition and gas developing was observed. The reaction mixture was allowed to stir at 0° C. for 1 h and then overnight at 20° C. Then 10% HCl (300 mL,) was added. The biphasic mixture obtained was extracted with AcOEt (300 mL). The organic layer was washed with water (300 mL, 6 vol) and then with 10% NaCl (300 mL). After solvent evaporation to dryness the residue was dissolved in IPA (200 mL) and re-distilled down to dryness. Then IPA (100 mL, 2 vol) and 2,6-Lutidine (17.5 mL) were added and the suspension refluxed for 20 min to obtain a clear dark solution. After cooling down to 20° C. the suspension was stirred overnight and then the solid filtered by washing upon the filter with water (200 mL). After drying at 40° C. under vacuum the product was obtained as beige solid in a 30.6% theoretical yield (22.13 g).

1H NMR (DMSO-d6) ppm: 11.29 (br.s., 1H); 8.21 (t, 1H); 7.90 (d, 1H); 7.75 (d, 1H); 7.15 (s, 1H)

### Preparation 3: 1(1R,5S/1S,5R)-[2-Fluoro-4-(trifluoromethyl)phenyl]-3 azabicyclo[3.1.0]hexane-2,4-dione

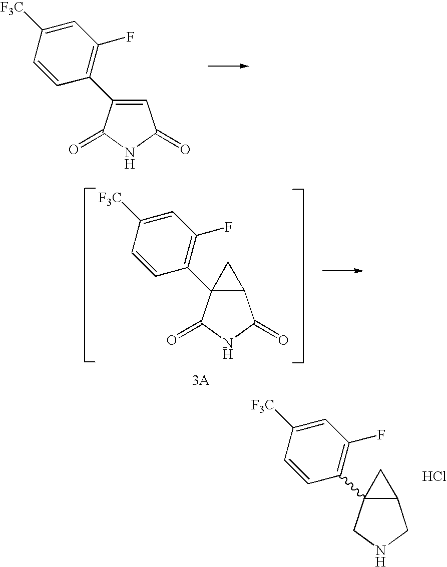

### Preparation 3A: 1 (1R,5S/1S,5R)-[2-Fluoro-4-(trifluoromethyl)phenyl]-3azabicyclo-[3.1.0]hexane-2,4-dione

Potassium hydroxide (258.1 g) was added to a stirred suspension of trimethylsulfoxonium iodide (1013 g) in dimethylsulfoxide (4470 mL) under N₂. The resulting mixture was allowed to stir at room temperature for 1 hr (or until a clear solution is observed).

3-[2-fluoro-4-(trifluoromethyl)phenyl]-1H-pyrrole-2,5-dione (prepared according to the method of Preparation 2, 596.0 g) dissolved in dimethylsulfoxide (1490 mL) was then added drop wise in 40 minutes keeping the internal temperature below 25° C. and the resulting mixture was allowed to stir at room temperature for 2 h.

The mixture was then diluted with tert-butyl methyl ether (6000 mL) and HCl 2N (4800 mL) was slowly added at room temperature. After separation of the two phases, the aqueous layer was extracted again with tert-butyl methyl ether (3000 mL) and the collected organic layers washed twice with water (3000 mL) and then with brine (3000 mL).

The organic layer was concentrated to 1800 mL then 4800 mL of tetrahydrofuran were added and the solution concentrated again to 1800 mL. The resulting tetrahydrofuran solution of the title compound was used as such in the following step.

### 1(1R,5S/1S,5R)-1-[2-Fluoro-4-(trifluoromethyl)phenyl]-3-azabicyclo[3.1.0]hexane salt of hydrochloric acid

NaBH₄ (351 g) was charged under N₂ followed by tetrahydrofuran (3600 mL) then the solution of 1-[2-Fluoro-4-(trifluoromethyl)phenyl]-3-azabicyclo[3.1.0]hexane in tetrahydrofuran prepared in the previous step was added dropwise in 1 h and the resulting suspension allowed to stir at room temperature for 1 hr.

BF3-THF complex (1440 mL) was then added dropwise in 1 h and 20 min keeping the internal temperature around 25° C. and the resulting suspension was stirred at 25° C. for 24 hrs.

The mixture was cooled down to 0° C. (internal) and methanol (2400 mL) was cautiously added in 2.5 h monitoring gas evolution. The suspension was then heated to reflux for 30 min and distilled down to 2400 mL at atmospheric pressure. The resulting suspension was diluted with tert-butyl methyl ether (6000 mL) and HCl 2 N (3600 mL) and the mixture was then stirred at room temperature for 10 minutes. The aqueous phase was discharged and the organic phase was washed twice with NaOH 2 N (about 3000 mL) and then with brine solution (3000 mL).

The organic phase was distilled down to 1800 mL then diluted with 3000 mL of tert-butyl methyl ether and again distilled down to 1800 mL.

3000 mL of tert-butyl methyl ether were added followed by 780 mL of HCl 5-6 N in isopropanol and the precipitation was immediately observed.

The suspension was aged overnight and then the solid filtered off washing with tert-butyl methyl ether (1200 mL). After drying at 40° C. for 24 h, the title compound (369.1 g) was obtained as a white solid in 57 mol % theoretical yield.

NMR (1H, DMSO-d6, δ ppm): 9.64 (bs, 2H); 7.70 (dd, 1H); 7.64 (t, 1H); 7.58 (dd, 1H); 3.62 (dd, 1H); 3.50 (dd, 1H); 3.42 (d, 1H); 3.35 (d, 1H); 2.24 (m, 1H); 1.41 (t, 1H); 1.15 (m, 1H)

### Preparation 4: (1S,5R)-1-[2-fluoro-4-(trifluoromethyl)phenyl]-3-azabicyclo[3.1.0]hexane salt of [(1R,4S)-7,7-dimethyl-2-oxobicyclo[2.2.1]hept-1-yl]methanesulfonic acid

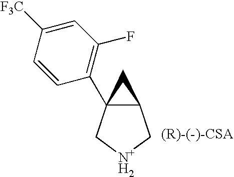

1-[2-fluoro-4-(trifluoromethyl)phenyl]-3-azabicyclo[3.1.0]hexane hydrochloride salt obtained from Preparation 3 (369.0 g) was suspended in tert-butyl methyl ether (2950 ml) and treated with NaOH 1N (1850 ml). The mixture was stirred for 5 minutes to achieve complete dissolution and then allowed to separate. The organic layer was washed twice with water (1850 ml) and then with 1850 ml of NaCl 10 % w/w solution. The organic layer was concentrated down to 1110 ml, diluted with more tert-butyl methyl ether (1850 ml) and distilled down to 1110 ml.

The solution was diluted with acetonitrile (1850 ml) and distilled down again to 1110 ml.

The resulting solution was diluted to 2960 ml and (−)-(R)-Camphorsulfonic acid was added (171.63 g). The exact amount of (−)-(R)-Camphorsulfonic acid was determined introducing a correction based on the assay w/w of the starting material.

Complete dissolution was observed followed after 30 minutes by precipitation. The slurry was aged for 22 hours at 20° C. under N₂; then filtered and the cake washed with additional acetonitrile (740 ml). The collected solid was placed in the oven at 40° C. under reduced pressure for 18 h. 223.5 g of the title compound were obtained in a 35.8% mol theoretical yield

1H NMR (DMSO-d6) ppm: 9.12 (br.s.; 2H); 7.72 (dd, 1H); 7.63 (t, 1H); 7.60 (m, 1H); 3.67 (dd, 1H); 3.56 (dd, 1H); 3.47 (d, 1H); 3.42 (d, 1H); 2.90 (d, 1H); 2.67 (m, 1H); 2.41 (d, 1H); 2.26 (m, 2H); 1.95 (t, 1H); 1.87 (m, 1H); 1.79 (d, 1H); 1.30 (m, 3H); 1.19 (m, 1H); 1.05 (s, 3H); 0.76 (s, 3H)

HPLC assay (short run): >99% a/a

HPLC chiral 1: enantiomeric excess (e.e.) >80%

### Preparation 5: Recrystallisation of (1S,5R)-1-[2-fluoro-4-(trifluoromethyl)phenyl]-3-azabicyclo[3.1.0]hexane salt of [(1R,4S)-7,7-dimethyl-2-oxobicyclo[2.2.1]hept-1-yl]methanesulfonic acid

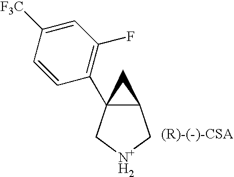

(1S,5R)-1-[2-fluoro-4-(trifluoromethyl)phenyl]-3-azabicyclo[3.1.0]hexane salt of [(1R,4S)-7,7-dimethyl-2-oxobicyclo[2.2.1]hept-1-yl]methanesulfonic acid obtained from Preparation 4 (223.5 g) was suspended in acetonitrile (1340 ml) at 20° C. under nitrogen. The suspension was heated to reflux for 90 minutes then allowed to cool down to ambient in 20 minutes and aged for additional 5 hours.

The suspension was filtered, washing with acetonitrile (447 ml). After drying at 40° C. for 18 hrs the title compound was obtained as a white solid in a 84.8 theoretical yield (189.5 g).

HPLC assay (short run): >99% a/a

HPLC chiral 1: enantiomeric excess (e.e.) >97%

### Preparation 6: (1R,5S/1S,5R)-1-[2-Fluoro-4-(trifluoromethyl)phenyl]-3-(3-{[4-methyl-1,3-oxazol-5-yl)-4H-1,2,4-triazol-3-yl]thio}propyl)-3-azabicyclo[3.1.0]hexane (as disclosed in WO 2005/080382)

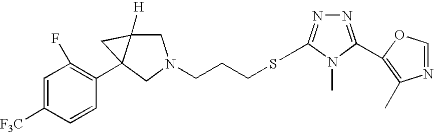

A mixture of (1R,5S/1S,5R)-1-[2-Fluoro-4-(trifluoromethyl)phenyl]-3-azabicyclo[3.1.0]hexane (700 mg, 2.8 mmol), 3-[(3-Chloropropyl)thio]-4-methyl-5-(4-methyl-1,3-oxazol-5-yl)-4H-1,2,4-triazole (3.4 mmol), Na₂CO₃ (3.4 mmol) and NaI (3.4 mmol) in DMF (anhydrous, 6 mL) was heated at 60° C. for 24 h. After elimination of the solvent under vacuo, the residue was dissolved in ethyl acetate and the organic layer was washed with saturated aqueous NaHCO₃ and dried over Na₂SO₄. This solution was filtered and the filtrate was concentrated in vacuo. The crude was purified by flash chromatography (dichloromethane to 10% MeOH in dichloromethane) to give 503 mg of the title compound.

NMR (¹H, CDCl₃): δ 7.89 (s, 1 H), 7.32-7.2 (m, 3H), 3.70 (s, 3H), 3.30 (t, 2H), 3.26 (dd, 1H), 3.10 (dd, 1H), 2.60 (t, 2H), 2.52 (dd, 1H), 2.51 (s, 3H), 2.43 (dd, 1H), 1.94 (m, 2H), 1.74 (m, 1H), 1.40 (t, 1H), 0.76 (dd, 1H). MS (m/z): 482.2[MH]⁺.

Preparation 6 was separated to give the separated enantiomers by semi-preparative HPLC using a chiral column Chiralpak AD 10 μm, 250×21 mm, eluent A: n-hexane; B: isopropanol +0.1 % isopropyl amine, gradient isocratic 9% B, flow rate 7 mL/min, detection UV at 200-400 nm. Retention times given were obtained using an analytical HPLC using a chiral column Chiralpak AD-H 5 μm, 250×4.6 mm, eluent A: n-hexane; B: isopropanol, gradient isocratic 15% B, flow rate 0.8 mL/min, detection UV at 200-400 nm.

Enantiomer 1 was recovered as white solid, Rt.=15.4 min.

Enantiomer 2 was recovered as white solid, Rt.=16.3 min.

Enantiomer 2 showed fpKi (D3)>1 log-unit higher than Enantiomer 1.

### Preparation 7 (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane hydrochloride (as disclosed in WO 2005/080382)

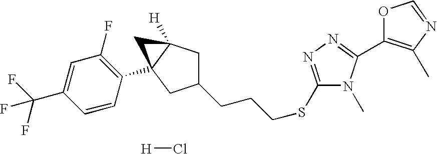

The free base of the title compound was prepared from (1S,5R)-1-[2-fluoro-4-(trifluoromethyl)phenyl]-3-azabicyclo[3.1.0]hexane. A mixture of (1S,5R)-1-[2-fluoro-4-(trifluoromethyl)phenyl]-3-azabicyclo[3.1.0]hexane (727 mg, 2.97 mmol), 3-[(3-Chloropropyl)thio]-4-methyl-5-(4-methyl-1,3-oxazol-5-yl)-4H-1,2,4-triazole (3.6 mmol.), K₂CO₃ (3.6 mmol.) and NaI (2.97 mmol) in DMF anhydrous was heated at 60° C. for 24 h. After elimination of the solvent under vacuo, the residue was dissolved in ethyl acetate and the organic layer was washed with saturated aqueous NaHCO₃ and dried over Na₂SO₄. This solution was filtered and the filtrate was concentrated in vacuo. The crude was purified by flash chromatography (dichloromethane to 10% MeOH in dichloromethane) to give 940 mg of the free base of the title compound.

This free base (886 mg) was converted to the hydrochloride salt (847 mg) by standard methods. The title compound was obtained as a white solid. Analytical Chiral HPLC confirmed the product to be identical to Enantiomer 2 of Preparation 6. NMR and MS data corresponded to those reported for Preparation 6.

The absolute configuration of the title compound was confirmed using comparative VCD and comparative OR analyses of the corresponding free base. Specific Optical Rotation of the corresponding free base: [α]_{D}=−42° (CDCl₃, T=25° C., c≈0.005 g/0.8 mL).

### Example 1

### (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate

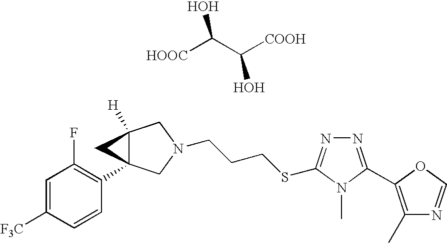

(1S,5R)-1-[2-fluoro-4-(trifluoromethyl)phenyl]-3-azabicyclo[3.1.0]hexane salt of [(1R,4S)-7,7-dimethyl-2-oxobicyclo[2.2.1]hept-1-yl]methanesulfonic acid (obtained from Preparation 5, 150.36 g) was suspended in tert-butylmethylether (1.5 L) at 20° C. An aqueous solution of 1M Sodium Hydroxide (0.75 L) was added and the mixture was stirred until complete dissolution. The phases were separated, and the organic one was washed twice with water (0.75 L each). After having discarded the aqueous phase, the solution was concentrated from 1,5 L to 0.45 L. tert-butylmethylether (0.75 L) was added and the mixture distilled again to 0.45 L (this operation was repeated twice). N-Methyl-Pyrrolidinone (0.6 L) was added and the solution concentrated to a volume of 0.6 L.

Potassium carbonate 325 mesh (69 g), potassium iodide (82.5 g), and 136.5 g of 3-[(3-chloropropyl)thio]-4-methyl-5-(4-methyl-1,3-oxazol-5-yl)-4H-1,2,4-triazole from Preparation 1, were added at 20° C. The mixture was then heated up to 55° C., and the heating was stopped after 8 hours. Ethyl acetate (1.2 L) and water (1.2 L) were added and the mixture was stirred until complete dissolution of the salts and then phases allowed separating. The aqueous phase was discarded and water (0.75 L) was added to wash the organic phase. The two phases were separated; the organic diluted with further ethyl acetate (0.3 L) and washed with water (0.75 L). The aqueous phase was discarded the organic one was concentrated to 0.6 L, diluted with additional ethyl acetate (0.75 L) and concentrate again to 0.6 L.

The mixture was treated with ethyl acetate (0.15 L) and methanol (0.3 L) before heating it up to 50° C. An aqueous solution of 47.25 g of L-tartaric acid dissolved in 0.15 L of water was added followed by authentic (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (L)-tartrate (salt), which has been prepared previously according to the present procedure (0.45 g). The mixture was cooled down to 20° C. and the precipitation started. The suspension was aged 4 hours, then the solid was filtrated and the cake washed three times with ethyl acetate (0.45 L) each wash. The product was dried in oven under vacuum at 40° C. for 4-20 hours. The title compound was obtained as an off-white solid was obtained in 79.4% theoretical yield (158 g).

HPLC assay (long run): 99.3% a/a

HPLC chiral 2: enantiomeric excess (e.e.) >98%

NMR (1H, DMSO-d6, δ ppm): 8.55 (s, 1H), 7.61 (d, 1H), 7.53 (m, 2H), 4.27 (s, 2H), 3.67 (s, 3H), 3.33 (d, 1H), 3.19 (t, 2H), 3.13 (d, 1H), 2.64 (t, 2H), 2.58 (dd, 1H), 2.50 (m, 1H), 2.37 (s, 3H), 1.94 (m, 1H), 1.86 (m, 2H), 1.35 (t, 1H), 0.82 (dd, 1H).

MS (m/z): 482[MH]⁺

### Example 2

### Further rework of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo-[3.1.0]hexane (2R,3R) tartrate

(1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane and (2R,3R) tartrate from previous stage (150 g) were suspended in 0.75 L of ethyl acetate and 0.3 L of methanol and heated up to 50° C. Once temperature has been reached, water (0.15 L) was added followed by authentic (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane salt of (L)-tartaric acid (prepared as discussed above, 0.45 g). The mixture was cooled to 20° C. over 30 min, and the precipitation started.

The suspension was aged 3.5 hours at 20° C., then the solid was filtrated and the cake washed twice with Ethyl Acetate (0.45 L each time).

The product was dried in oven under vacuum at 40° C. for 4-20 hours. The title compound was obtained as a white solid in 87% theoretical yield (129.7 g).

HPLC assay (long run): 99.7% a/a

HPLC chiral 2: enantiomeric excess (e.e.) >98%

NMR (1H, DMSO-d6, δ ppm): 8.55 (s, 1H), 7.61 (d, 1H), 7.53 (m, 2H), 4.27 (s, 2H), 3.67 (s, 3H), 3.33 (d, 1H), 3.19 (t, 2H), 3.13 (d, 1H), 2.64 (t, 2H), 2.58 (dd, 1H), 2.50 (m, 1H), 2.37 (s, 3H), 1.94 (m, 1H), 1.86 (m, 2H), 1.35 (t, 1H), 0.82 (dd, 1H).

MS (m/z): 482[MH]⁺

A sample from this preparation has been analysed by using the following conditions:

X-Ray Powder Diffraction

X Ray Powder Diffraction (XRPD) analysis was performed on Siemens D5005, using Sol-X detector. The acquisition conditions were: radiation: Cu Kα, generator tension: 40 kV, generator current: 50 mA, start angle: 2.0 °2Θ, end angle: 45.0 °2Θ, step size: 0.02 °2Θ, time per step: 0.5 seconds. The sample was prepared on a low background sample holder.

It will be recognised that spectra and diffraction data will vary slightly according to various factors such as the temperature, concentration and instrumentation used. The skilled person will recognise that XRPD peak positions are affected by differences in sample height. The peak positions quoted herein are thus subject to a variation of ±0.15 degrees 2-theta.

Raman Spectroscopy

Instrument Configuration: Kaiser RXN1 Kaiser Optical System Micro Raman. Sample on Al sample pan at 4 cm⁻¹ resolution, laser λ=785 nm, power output 100 mw.

Differential Scanning Calorimetry (DSC)

Instrument configuration: TA Q1000, hermetic sample pan, run@10K/min, N₂ Flow=30 mL/min.

It should be recognized that the endotherm peak as measured is dependent under a number of factors including the machine employed, the rate of heating, the calibration standard, humidity and the purity of the sample used.

The analytical results suggested that (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl]-3-azabicyclo-[3.1.0]hexane (2R,3R) tartrate is a solvate containing 1.5 molecules of water. The result was subsequently confirmed by a single crystal X-ray analysis of a sample prepared in an analogous way to that described above. Such single crystal had been suitably produced by growing up the crystal through addition of water at raised temperature. In particular, that sample has been suspended in a mixture ethyl acetate and MeOH then heated until it was solubilised at 50° C./500 rpm. To this solution H₂O has been added. After 1 h stirring the precipitation occurred and then the suspension has been cooled to 20° C. (in 15 min) and stirred for 3 h and then filtered. The solid was then dried at 40° C. under vacuum overnight.

From the single crystal X-ray analysis results that asymmetric unit of (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate contains a cation and a hydrogen tartrate anion, together with 1.5 molecules of water. The half water molecule is positioned on a crystallographic two-fold axis and it is shared between two asymmetric units.

XRPD Angles and D Spacings for a Sample from this Preparation:

| Angle | d value |
|---|---|
| 2-Theta° | Angstrom |
| 5.9 | 15.0 |
| 6.9 | 12.9 |
| 7.7 | 11.4 |
| 10.2 | 8.7 |
| 11.8 | 7.5 |
| 11.9 | 7.4 |
| 13.4 | 6.6 |
| 14.6 | 6.1 |
| 15.1 | 5.8 |
| 15.5 | 5.7 |
| 15.7 | 5.7 |
| 16.4 | 5.4 |
| 17.1 | 5.2 |
| 17.6 | 5.0 |
| 18.4 | 4.8 |
| 19.3 | 4.6 |
| 19.5 | 4.5 |
| 19.9 | 4.5 |
| 20.4 | 4.3 |
| 20.6 | 4.3 |
| 22.7 | 3.9 |
| 23.6 | 3.8 |
| 24.5 | 3.6 |
| 24.7 | 3.6 |
| 24.9 | 3.6 |
| 25.2 | 3.5 |
| 26.3 | 3.4 |
| 26.5 | 3.4 |
| 27.0 | 3.3 |
| 27.3 | 3.3 |
| 27.5 | 3.2 |
| 27.9 | 3.2 |
| 28.5 | 3.1 |
| 29.8 | 3.0 |
| 30.5 | 2.9 |
| 31.3 | 2.9 |
| 32.3 | 2.8 |
| 34.1 | 2.6 |
| 35.7 | 2.5 |
| 36.1 | 2.5 |
| 36.9 | 2.4 |
| 39.0 | 2.3 |
| 39.2 | 2.3 |
| 39.9 | 2.3 |

Description of Figures:

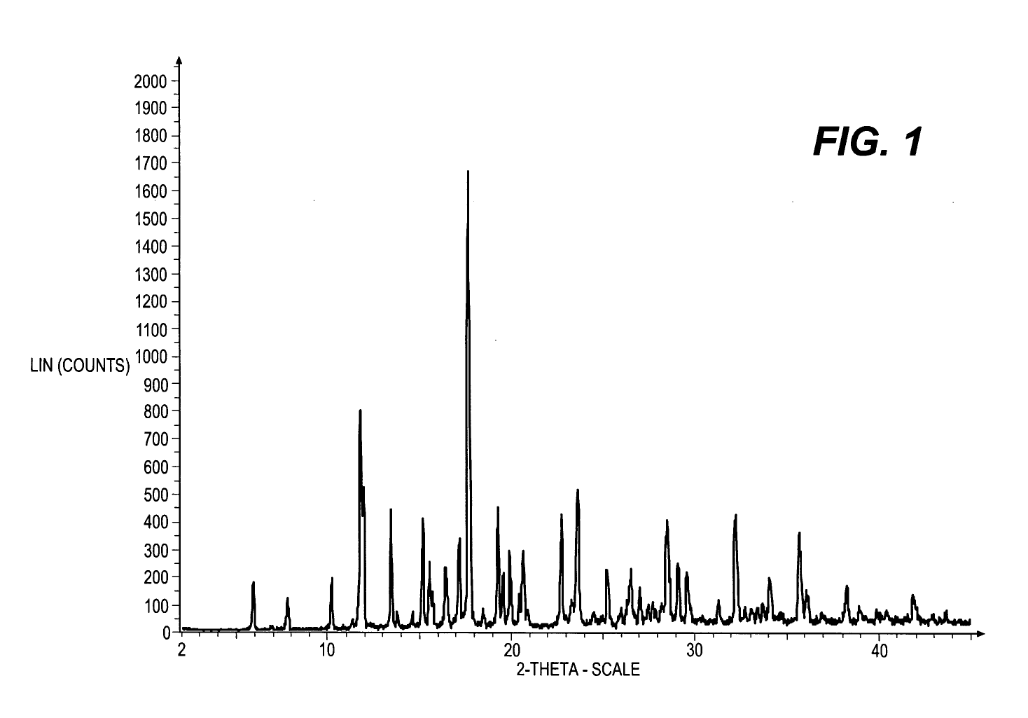

FIG. 1 shows X-Ray powder diffraction data obtained for (1S,5R)-1-[2-fluoro-4-(trifluoromethyl)phenyl]-3-(3-{[4-methyl-5-(4-methyl-1,3-oxazol-5-yl)-4H-1,2,4-trizaol-3-yl]thio}propyl)-3-azabicyclo[3.1.0]hexane (2R,3R) tartrate as herein described.

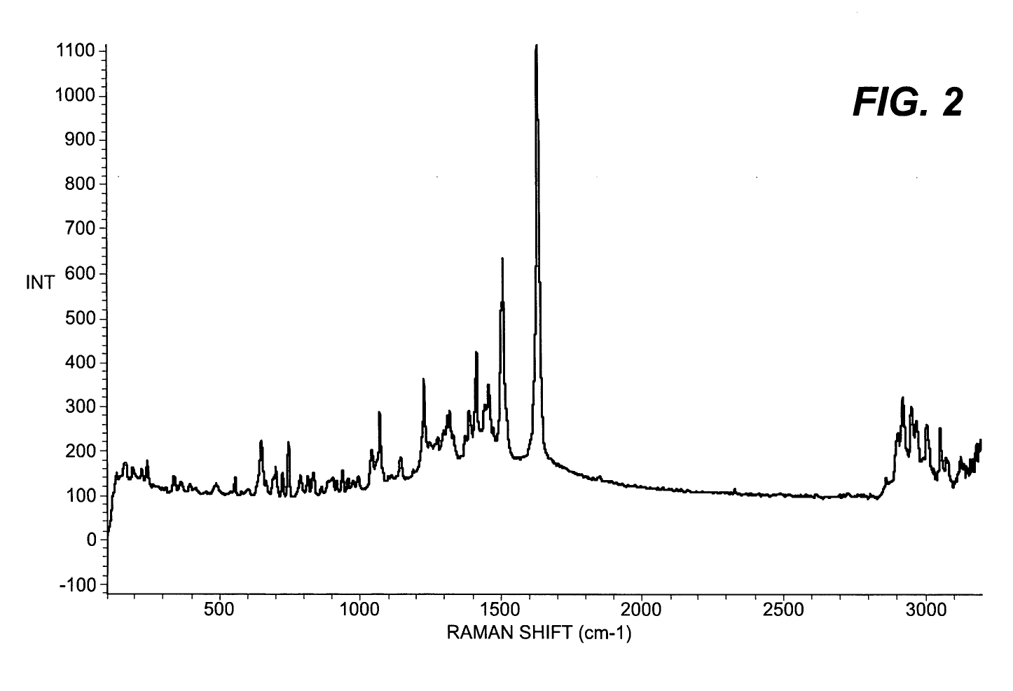

FIG. 2 shows the Raman spectrum of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described.

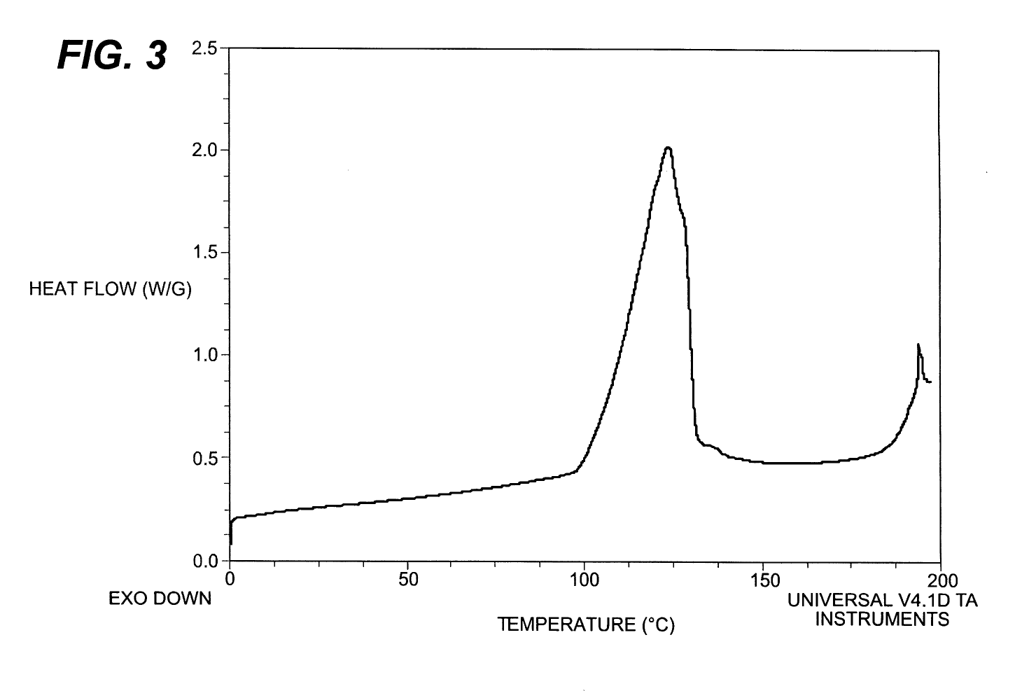

FIG. 3 shows a Differential Scanning Calorimetry (DSC) thermogram of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described.

### Example 3

### Alternative preparation of (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate

(1S,5R)-1-[2-fluoro-4-(trifluoromethyl)phenyl]-3-azabicyclo[3.1.0]hexane salt of [(1R,4S)-7,7-dimethyl-2-oxobicyclo[2.2.1]hept-1-yl]methanesulfonic acid (310 g), prepared in an analogous way as described before in Preparation 4, was suspended in tert-butylmethyl ether (3.1 L) and treated with NaOH 1N (1.55 L). After phase separation the organic layer was washed twice with water (1.55 L each) and then evaporated down to about 620 mL. Fresh tert-butylmethylether (620 mL) was added and the solution evaporated down again to 620 mL. After addition of DMF (0.93 L), the solution was evaporated down to about 0.93 L. K₂CO₃ 325 mesh (143 g), KI (171 g) and 3-[(3-chloropropyl)thio]-4-methyl-5-(4-methyl-1,3-oxazol-5-yl)-4H-1,2,4-triazole (283 g) prepared in analogy with Preparation 1 were added at room temperature. The obtained suspension was then warmed at 62-63° C. for 5 h and then cooled down to 20° C. After dilution with ethyl acetate (1.55 L), water (1.55 L) was added and phases allowed separating. The organic layer was washed twice with water (775 mL each), diluted with further ethyl acetate (0.31 L), concentrated to 620 mL, diluted with additional ethyl acetate (620 mL) and evaporated down again to dryness. A portion of the so obtained yellow waxy solid (315 g over a total of 330 g) was dissolved in acetone (2.30 L) and L-Tartaric Acid (93.3 g) was added at 20° C. After 20 min water (74 mL) was added to dissolve completely the acid. Precipitation of a white solid immediately occurred. The mixture was stirred for 3h at 20° C., then filtered and the cake washed with acetone/water 2/1 mixture (0.9 L). After drying under vacuum at 40° C. for 20 h, the title compound was obtained as an off-white solid (347 g) and 97.8% a/a typical purity by HPLC (short run).

NMR (1H, DMSO-d6): 8.55 (s, 1H), 7.61 (d, 1H), 7.53 (m, 2H), 4.27 (s, 2H), 3.67 (s, 3H), 3.33 (d, 1H), 3.19 (t, 2H), 3.13 (d, 1H), 2.64 (t, 2H), 2.58 (dd, 1H), 2.50 (m, 1H), 2.37 (s, 3H), 1.94 (m, 1H), 1.86 (m, 2H), 1.35 (t, 1H), 0.82 (dd, 1H).

A sample from this preparation has been analysed in the same conditions as disclosed in Example 2 and here are the corresponding data:

XRPD Angles and D Spacings for a Sample from this Preparation:

| Angle | d value |
|---|---|
| 2-Theta° | Angstrom |
| 5.9 | 15.0 |
| 6.9 | 12.8 |
| 7.8 | 11.4 |
| 10.2 | 8.7 |
| 11.8 | 7.5 |
| 11.9 | 7.4 |
| 13.5 | 6.6 |
| 14.6 | 6.1 |
| 15.1 | 5.8 |
| 15.5 | 5.7 |
| 15.7 | 5.7 |
| 16.5 | 5.4 |
| 17.1 | 5.2 |
| 17.6 | 5.0 |
| 18.4 | 4.8 |
| 19.3 | 4.6 |
| 19.5 | 4.5 |
| 19.9 | 4.4 |
| 20.4 | 4.3 |
| 20.6 | 4.3 |
| 22.7 | 3.9 |
| 23.7 | 3.8 |
| 24.4 | 3.6 |
| 24.7 | 3.6 |
| 25.0 | 3.6 |
| 25.3 | 3.5 |
| 25.8 | 3.4 |
| 26.3 | 3.4 |
| 26.5 | 3.4 |
| 27.3 | 3.3 |
| 27.5 | 3.2 |
| 27.9 | 3.2 |
| 28.5 | 3.1 |
| 29.8 | 3.0 |
| 30.4 | 2.9 |
| 31.4 | 2.8 |
| 32.3 | 2.8 |
| 32.8 | 2.7 |
| 34.1 | 2.6 |
| 35.7 | 2.5 |
| 36.1 | 2.5 |
| 36.9 | 2.4 |
| 39.0 | 2.3 |
| 39.2 | 2.3 |
| 39.8 | 2.3 |

Description of Figures:

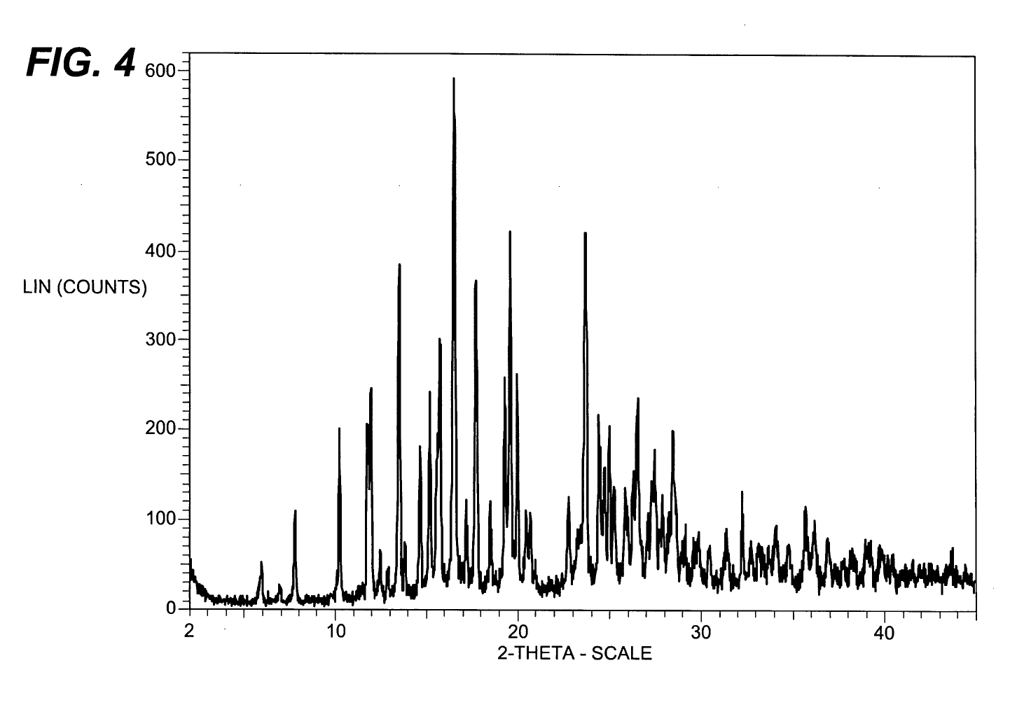

FIG. 4 shows X-Ray powder diffraction data obtained for (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described and in the conditions described in Example 2.

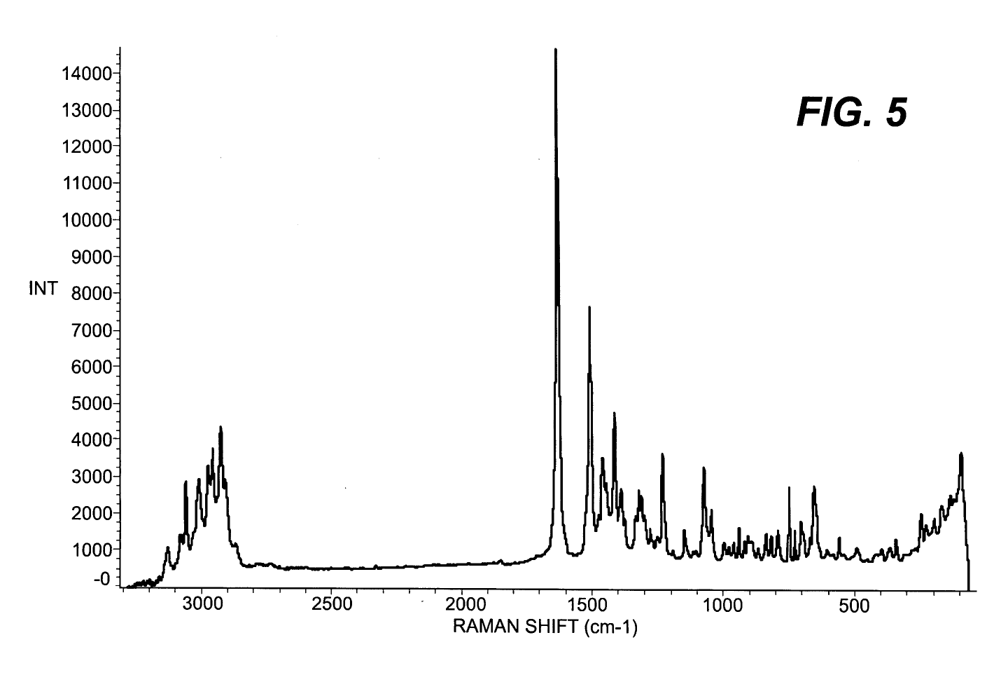

FIG. 5 shows the Raman spectrum of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described and in the conditions described in Example 2.

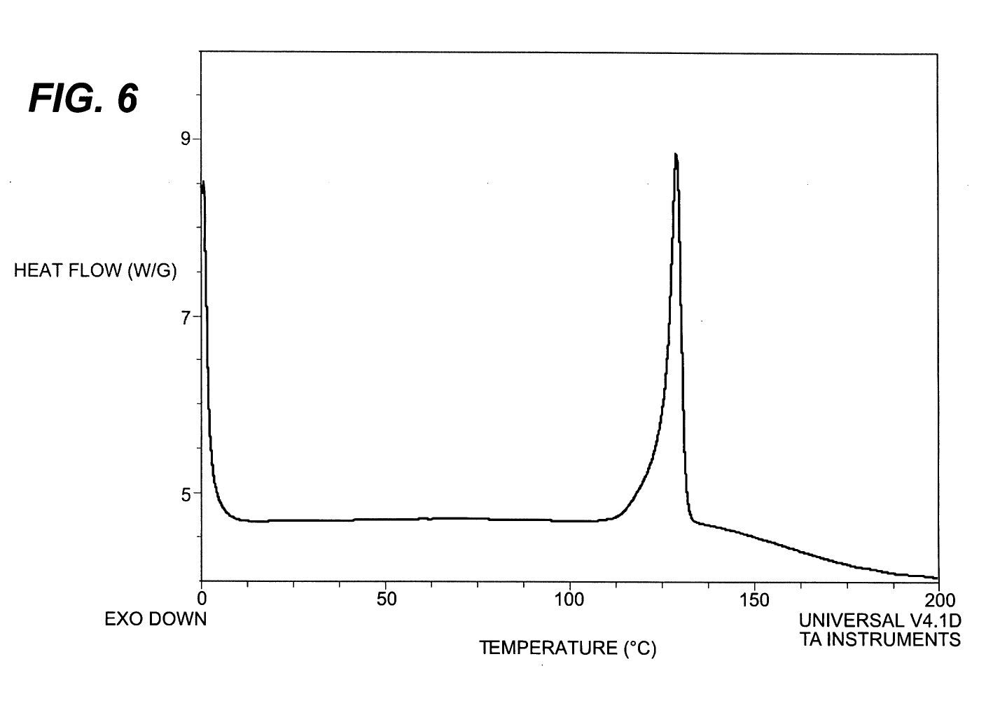

FIG. 6 shows a Differential Scanning Calorimetry (DSC) thermogram of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described registered with a different instrument from the one described in Example 2.

Instrument configuration: PE DSC 7, closed sample pan, run@10K/min, N2 Flow=30 mL/min. It should be recognized that the endotherm peak as measured is dependent under a number of factors including the machine employed, the rate of heating, the calibration standard, humidity and the purity of the sample used.

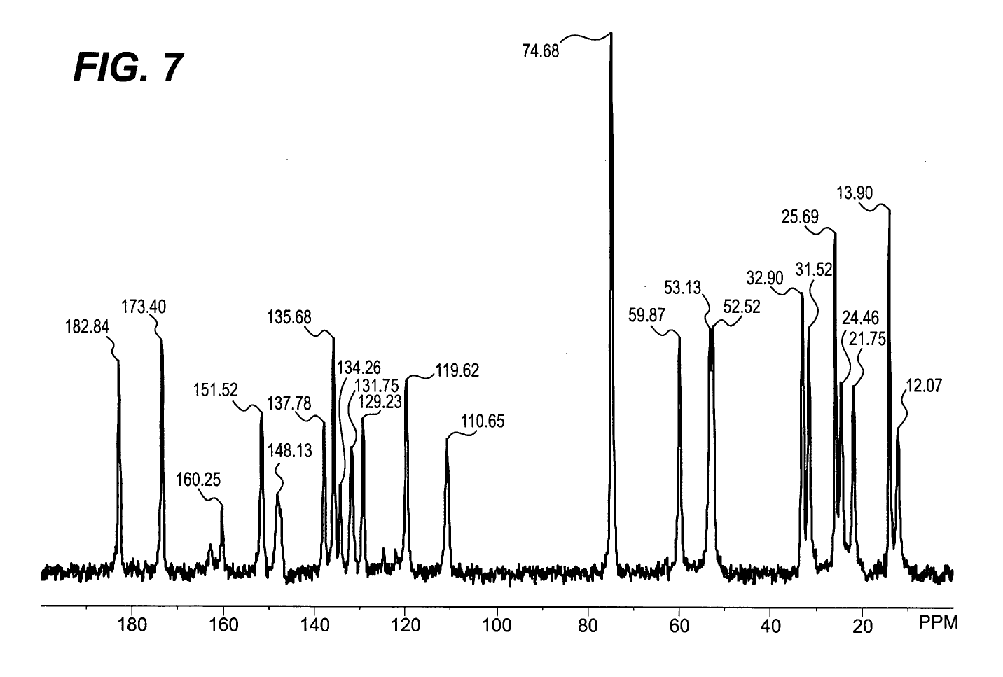

FIG. 7 shows a Carbon-13 solid State NMR spectrum performed on a different sample of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate but prepared in an analogous way as herein described.

Carbon-13 solid-state NMR (SSNMR) data was acquired using a Bruker Av400 spectrometer operating at a proton frequency of 399.87 MHz. A 4-mm Bruker HFX MAS (magic-angle spinning) probe was used. Samples were gently packed into a zirconia rotor and spun at 8 kHz. Data was obtained using ramped cross-polarization and a TOSS (total sideband suppression) pulse sequence. Proton decoupling was performed at an RF power of 100 kHz using the SPINAL64 decouping sequence. Characteristic carbon-13 NMR peak positions are reported in parts per million (ppm) frequency relative to tetramethylsilane at 0 ppm, and have a precision of ±0.3 ppm caused by instrumental variability and calibration.

(1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate as herein described is characterised by a solid state Carbon 13 spectrum NMR having resonances at 182.9, 173.4, 151.6, 137.7, 135.6, 129.3, 119.5, 74.6, 59.8, 32.9, 31.5, 25.7, 21.7,13.9 ±0.3 ppm

### Example 4

### Stability of (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate and hydrochloride

The drug substances ((1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate and hydrochloride, prepared in an analogous way as described before in Example 3 and Preparation 7, respectively) were packaged in amber glass vials under air atmosphere, 6 mL volume, closed with Teflon coated plugs, stored upright.

The solid state accelerated condition adopted were 40° C./75% RH (Relative Humidity) closed and exposed, and 50° C./amb RH (Ambient Relative Humidity) closed under air atmosphere.

At one month time point the following samples have been analysed for the appearance, assay and total impurities.

Assay and total impurities tests have been performed by HPLC with fast gradient method.

The chromatographic conditions are:

| Column type: | Phenomenex LUNA C18 (2) | | |
|---|---|---|---|
| Column length (cm): | 5 | | |
| Internal diameter (cm): | 0.21 | | |
| Particle Size (μm): | 3 | | |
| Mobile phase: | A: 0.05% v/v TFA in WATER/ | | |
| | B: 0.05% v/v TFA In | | |
| | Acetonitrile | | |
| Step-1 Time-Reserv.A-Reserv.B: | Time 0 min | 100% | A |
| Step-2 Time-Reserv.A-Reserv.B | Time 8 min | 5% | A |
| Step-3 Time-Reserv.A-Reserv.B | Time 8.01 min | 100 | A |

| | |
|---|---|
| Flow rate (mL/min): | 1 |
| Column temperature [° C.]: | 40 |
| Detector type: | UV |
| Wavelenght (nm): | 220 |
| Injection volume (μL): | 2 |
| Typical retention time: | 3.9 min |

The stability data after 1 month resulted favourable for the tartrate salt, as a value of total impurities around 0.5% a/a was found at any stability condition tested.

An increase of total impurities has been observed for the hydrochloride salt at any investigated stability condition.

| | | | | |
|---|---|---|---|---|
| Time point and storage | | | | |
| condition | Appearance | % w/w (salt) | % on initial | Tot. Imp. % a/a |

(1S,5R)-1-[2-Fluoro-4-(trifluoromethyl)phenyl]-3-(3-{[4-methyl-5-(4-methyl-1,3-oxazol-5-yl)-4H-
1,2,4-triazol-3-yl]thio}propyl)-3-azabicyclo[3.1.0]hexane (2R,3R) tartrate salt
1 month stability and 3 months stability

| | | | | |
|---|---|---|---|---|
| INITIAL | white powder | 101.4 | — | 0.442 |
| 1 month 40° C./75% RH | unchanged | 101.5 | 100.1 | 0.508 |
| exposed | | | | |
| 1 month 40° C./75% RH closed | unchanged | 101.3 | 99.9 | 0.517 |
| 1 month 50° C./RH amb closed | unchanged | 102.2 | 100.8 | 0.645 |
| 3 months 40° C./75% RH | unchanged | 110.6 | 109.1 | 2.24 |
| exposed | | | | |
| 3 months 40° C./75% RH | unchanged | 118.1 | 116.5 | 4.44 |
| closed | | | | |
| 3 months 50° C./RH amb | unchanged | 102.9 | 100.7 | 1.280 |
| closed | | | | |

(1S,5R)-1-[2-Fluoro-4-(trifluoromethyl)phenyl]-3-(3-{[4-methyl-5-(4-methyl-1,3-oxazol-5-yl)-4H-
1,2,4-triazol-3-yl]thio}propyl)-3-azabicyclo[3.1.0]hexane hydrochloride salt
1 month stability and 3 months stability

| | | | | |
|---|---|---|---|---|
| INITIAL | white powder | 102.1 | — | 1.197 |
| 1 month 40° C./75% RH | unchanged | 93.8 | 91.9 | 2.985 |
| exposed | | | | |
| 1 month 40° C./75% RH closed | unchanged | 92.3 | 90.4 | 3.697 |
| 1 month 50° C./RH amb closed | unchanged | 91.8 | 89.9 | 4.091 |
| 3 months 40° C./75% RH | Off white | 74.9 | 73.4 | 1.310 |
| exposed | powder | | | |
| 3 months 40° C./75% RH | Off white | 88.6 | 86.7 | 4.510 |
| closed | powder | | | |
| 3 months 50° C./RH amb | Off white | 85.7 | 84.0 | 7.690 |
| closed | powder | | | |

Then, it is evident to the skilled person that Tartrate salt shows an improved stability in comparison with the Hydrochloride.

### Example 5

### (1S,5R)-1-[2-Fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate capsules

The following formulation example is illustrative only and it is not intended to limit the scope of the present invention.

Hard capsules of the title compound are white, opaque, containing 5 mg, and 25 mg of the corresponding free base of the title compound (as the L-tartrate sesquihydrate salt).

Capsules composition

| | | |
|---|---|---|
| | Quantity | |
| Component | mg/capsule | Function |

| | | | |
|---|---|---|---|
| Title compound¹ | 6.75² | 33.73³ | Active |
| Pregelatinized Starch | 310.85 | 264.02 | Diluent |
| Colloidal Silica Dioxide | 0.80 | 0.75 | Glidant |
| Magnesium Stearate | 1.60 | 1.50 | Lubricant |
| Total | 320.00 | 300.00 | — |
| (Capsule Fill weight) | | | |
| Hypromellose Capsule | one | one | |
| Shell⁴ | | | |

Notes:
¹The quantity of the title compound may be adjusted to reflect the assigned purity of the input drug substance
²Corresponding to 5 mg as free base of the title compound
³Corresponding to 25 mg as free base of the title compound
⁴White, opaque, size 0, hard hypromellose capsule shells.

The formulation may be changed in compliance with reasonable variations provided.

The claims of such subsequent application may be directed to any novel feature or combination of features relating to the invention described herein. They may take the form of product, process or use claims and may include, by way of example and without limitation, the claims that follow.

1. 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate or a pharmaceutically acceptable solvate thereof.

2. (1S,5R)-1-[2-fluoro-4-(trifluoromethyl)phenyl]-3-(3-{[4-methyl-5-(1,3-oxazol-5-yl)-4H-1,2,4-triazol-3-yl]thio}propyl)-3-azabicyclo[3.1.0]hexane tartrate or a pharmaceutically acceptable solvate thereof.

3. (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate or a pharmaceutically acceptable solvate thereof.

4. A compound as claimed in claim 1, 2 or 3 wherein the ratio of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane or (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane to tartaric acid (by mole) is 1:1.

5. A compound according to claim 2 wherein the ratio of (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane to (2R,3R) tartaric acid (by mole) is 1:1.

6. A compound according to claim 1 which is a hydrate.

7. A compound according to claim 2 which is a hydrate.

8. A compound according to claim 3 which is a hydrate.

9. A compound according to claim 1 which is a sesquihydrate.

10. A compound according to claim 2 which is a sesquihydrate.

11. A compound according to claim 3 which is a sesquihydrate.

12. A compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate having a differential scanning calorimetry thermogram substantially as shown in FIG. 2, wherein the DSC was performed at a scan rate of 10K per minute.

13. A crystalline compound according to claim 14 having a differential scanning calorimetry thermogram with an onset at about T=122° C.

14. A crystalline compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate having an X-ray powder diffraction spectrum substantially as shown in FIG. 1, wherein the XRD pattern is expressed in terms of 2 theta angles and obtained with a diffractomer using Copper Kα radiation:

| Angle | d value |
|---|---|
| 2-Theta° | Angstrom |
| 5.9 | 15.0 |
| 6.9 | 12.9 |
| 7.7 | 11.4 |
| 10.2 | 8.7 |
| 11.8 | 7.5 |
| 11.9 | 7.4 |
| 13.4 | 6.6 |
| 14.6 | 6.1 |
| 15.1 | 5.8 |
| 15.5 | 5.7 |
| 15.7 | 5.7 |
| 16.4 | 5.4 |
| 17.1 | 5.2 |
| 17.6 | 5.0 |
| 18.4 | 4.8 |
| 19.3 | 4.6 |
| 19.5 | 4.5 |
| 19.9 | 4.5 |
| 20.4 | 4.3 |
| 20.6 | 4.3 |
| 22.7 | 3.9 |
| 23.6 | 3.8 |
| 24.5 | 3.6 |
| 24.7 | 3.6 |
| 24.9 | 3.6 |
| 25.2 | 3.5 |
| 26.3 | 3.4 |
| 26.5 | 3.4 |
| 27.0 | 3.3 |
| 27.3 | 3.3 |
| 27.5 | 3.2 |
| 27.9 | 3.2 |
| 28.5 | 3.1 |
| 29.8 | 3.0 |
| 30.5 | 2.9 |
| 31.3 | 2.9 |
| 32.3 | 2.8 |
| 34.1 | 2.6 |
| 35.7 | 2.5 |
| 36.1 | 2.5 |
| 36.9 | 2.4 |
| 39.0 | 2.3 |
| 39.2 | 2.3 |
| 39.9 | 2.3 |

15. A crystalline compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate having an X-ray powder 10 diffraction spectrum which comprises the following peaks expressed in 2-theta values: 5.9±0.15, 6.9±0.15, 10.2±0.15, 11.8±0.15, 11.9±0.15, 16.4±0.15, 17.6±0.15; and obtained with a diffractometer using Copper Kα radiation.

16. A crystalline compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1 S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate having substantially the same carbon-13 solid-state nuclear magnetic resonance (SSNMR) spectrum for as FIG. 7 wherein the spectrum was acquired on a spectrometer operating at a proton frequency of 399.87 MHz, a spinning speed of 8 kHz.

17. A crystalline compound according to claim 16 having substantially the same carbon-13 solid-state nuclear magnetic resonance (SSNMR) spectrum for as FIG. 7 wherein the spectrum was acquired on a spectrometer operating at a proton frequency of 399.87 MHz, a spinning speed of 8 kHz, wherein the SSNMR exhibits resonances at 182.9, 173.4, 151.6, 137.7, 135.6, 129.3, 119.5, 74.6, 59.8, 32.9, 31.5, 25.7, 21.7, 13.9±0.3 ppm.

18. A crystalline compound compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate having substantially the same carbon-13 solid-state nuclear magnetic resonance (SSNMR) spectrum for as FIG. 7 wherein the spectrum was acquired on a spectrometer operating at a proton frequency of 399.87 MHz, a spinning speed of 8 kHz, wherein the SSNMR exhibits resonances at 182.9, 173.4, 151.6, 59.8, 25.7, 21.7, 13.9±0.3 ppm.

19. A pharmaceutical composition comprising a compound selected from the group consisting of 1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane tartrate, and (1S,5R)-1-[2-fluoro-4-(trifluoromethyl)phenyl]-3-(3-{[4-methyl-5-(4-methyl-1,3-oxazol-5-yl)-4H-1,2,4-triazol-3-yl]thio}propyl)-3-azabicyclo[3.1.0]hexane (2R,3R) tartrate 1-16 and a pharmaceutically acceptable carrier.
